(19) United States
(12) Patent Application Publication
(10) Pub. No.: US 2010/0239500 A1
(43) Pub. Date: Sep. 23, 2010

(54) SUBSTITUTED TRIAZINE COMPOUNDS FOR NERVE REGENERATION

(76) Inventors: John L. Bixby, Miami, FL (US); Vance P. Lemmon, Miami, FL (US); Young-Tae Chang, Singapore (SG); Jae-Wook Lee, San Diego, CA (US); Jaeki Min, Marietta, GA (US); Lynn Usher, Cali (CO)

Correspondence Address: VENABLE LLP, P.O. BOX 34385, WASHINGTON, DC, 20043-9998, US

(21) Appl. No.: 12/594,066

(22) PCT Filed: Mar. 31, 2008
(86) PCT No.: PCT/US08/04168

§ 371 Date: Jun. 1, 2010

Related U.S. Application Data
(60) Provisional application No. 60/920,811, filed on Mar. 30, 2007.

(51) Int. Cl. A61K 49/00; C07D 403/12; C07D 403/14; C40B 40/04; A61K 31/53; C40B 30/06; A61P 25/00

(52) U.S. Cl. 424/9.2; 544/212; 506/15; 514/245; 506/10

(57) ABSTRACT

A family of substituted triazine compounds is synthesized by combinatorial solid phase chemistry. These compounds were found to increase the growth of neurons/axons from central nervous system neurons that had been damaged, and can be used in methods and pharmaceutical compositions for treating injuries, diseases and conditions associated with nerve damage.

This application claims priority to U.S. provisional application No. 60/920,811, filed Mar. 30, 2007, which is hereby incorporated by reference.

### FIELD OF THE INVENTION

The present application relates to substituted triazine compounds that have been found to promote nerve regeneration, and methods of use.

### BACKGROUND

Traumatic brain injury, stroke, spinal cord injury, multiple sclerosis, and medical problems from diseases that affect the central nervous system or optic nerve, such as Parkinson's Disease, Alzheimer's Disease, or glaucoma, all result from damage to or severing of axons in the central nervous system. Damage to the adult central nervous system (CNS) often leads to persistent deficits because of the inability of mature axons to regenerate after injury. Chemical compounds that could be administered to patients to aid in axon regeneration and/or sprouting would be useful in all of the above situations.

The nervous system has the remarkable ability to adapt and respond to various stimuli ranging from physiological experiences associated with learning and memory, to pathological insults such as traumatic injury, stroke, or neurodegenerative disease. In addition to plasticity at the functional level, the response of the nervous system might also take the form of structural remodeling. Neural injury is often accompanied by a transient period of anatomic remodeling in the form of local sprouting at the lesion site. However, although mature CNS neurons can survive for years after axotomy, the severed axons ultimately fail to regenerate beyond the lesion site, in contrast to those in the peripheral nervous system or the embryonic nervous system.

The regeneration failure in the adult CNS might be partly attributed to the gradual decline in the intrinsic growth ability of neurons as an animal matures. After injury, the ends of lesioned axons became swollen into ‘dystrophic endbulbs’, which remain at or near the lesion site without migrating forward. Although it was previously believed that these endbulbs are quiescent, recent studies suggest that these dystrophic endings are not quiescent at all, but are highly active structures that might be capable of regeneration with appropriate stimulation. In fact, some injured axons retain a limited capacity for regrowth, and can extend over long distances in the permissive environment of a peripheral nerve graft. Furthermore, neurons such as those in dorsal root ganglia have axons in both the CNS and the PNS, but can regenerate only their peripheral processes. These observations suggest that interactions with different environments contribute to the differential regenerative responses.

Increasing evidence suggests that many inhibitory or repulsive guidance cues involved in axon pathfinding during development actually persist into adulthood and might restrict axon regeneration after injury. The myelin structure formed by oligodendrocytes, which normally ensheathes nerve fibers, can be damaged by injury, exposing severed axons to myelin-associated inhibitors. In addition, reactive astrocytes form a glial scar at the lesion site, and act as an additional barrier to axon regrowth.

Among the molecular inhibitors of the adult CNS glial environment are chondroitin sulfate proteoglycans (CSPGs) associated with reactive astrocytes from the glial scar and myelin-associated inhibitors from intact oligodendrocytes and myelin debris. Numerous myelin-associated components that can inhibit axon outgrowth in vitro have been identified, including Nogo, myelin-associated glycoprotein, oligodendrocyte myelin glycoprotein, the transmembrane semaphorin 4D, and ephrin B3. Because of the remarkable diversity among these myelin components, their respective contributions to myelin inhibition remain unclear.

Another important source of inhibition of axon regeneration is the glial scar that forms after CNS injury. The glial reaction to injury results in the recruitment of microglia, oligodendrocyte precursors, meningeal cells and astrocytes to the lesion site, resulting in scar formation. These responses may in part be beneficial, because they isolate the injury site and minimize the area of inflammation and cellular degeneration. However, many astrocytes in the injured area become hypertrophic and adopt a reactive phenotype, releasing inhibitory extracellular matrix molecules known as chondroitin sulfate proteoglycans (CSPGs). After injury, CSPG expression is rapidly upregulated by reactive astrocytes, forming an inhibitory gradient that is highest at the center of the lesion and diminishes gradually into the penumbra.

Besides CSPGs there are other known and unknown inhibitory elements of the glial scar. It is clear that there are many inhibitory molecules in the adult CNS environment that might be responsible for regenerative failure after injury. To some extent, these molecular inhibitors are distinct from the trophic and guidance cues that regulate the initial formation of the nervous system. Instead, they are mainly associated with the later states of nervous system development, including myelin formation and termination of the critical period for experience-drive plasticity. During CNS injury, damaged axons might be initially exposed to various myelin-associated inhibitors from oligodendrocytes and myelin debris. Over time, reactive astrocytes are recruited to the glial scar, releasing inhibitory CSPGs that further prevent axon repair. As a highly overlapping set of mechanisms limits both axon repair after injury and local plasticity in the intact adult, alleviating these inhibitory influences might not only promote the regrowth of damaged axons, but might also enhance compensatory sprouting from preserved fibers.

There are no existing therapies that promote CNS axon regeneration in humans.

Currently, treatment options for CNS injury remain limited to minimizing inflammation and swelling in the acute setting to preserve intact fibers, and physical therapy in the long term to stimulate the little plasticity that is available in adults. Attempts to promote axon repair by neutralizing endogenous inhibitory mechanisms could potentially shift the current treatment from palliative care to actual restoration of function. In the absence of long-distance regeneration, even a small improvement in compensatory sprouting and local plasticity could translate to significant improvement in clinical outcomes.

### SUMMARY

Studies described herein have demonstrated that substituted triazines, which are relatively small molecules, function to increase axonal regeneration in vitro and in vivo as well. These compounds should be useful, inter alia, in pharmaceutical compositions for treatment of conditions associated with nerve damage. Compound libraries comprising a plurality of the compounds can be assembled and screened to identify compounds effective for such treatment. Furthermore, the compounds may also be useful in identifying molecular mechanisms underlying inhibition of CNS axon growth.

The novel trisubstituted triazine compounds found to promote nerve regeneration have the following formula:

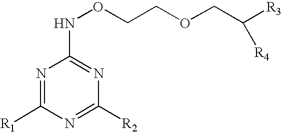

wherein R₁ is substituted or unsubstituted alkyl or aryl/alkyl group and R₂ is a substituted or unsubstituted amine; R₃ and R₄ are independently hydrogen, substituted or unsubstituted amine, substituted or unsubstituted C₁₋₁₀ alkyl, C₂₋₁₀ alkene, and C₂₋₂₀ alkynyl.

### BRIEF DESCRIPTION OF THE DRAWINGS

FIG. 1 is a synthetic scheme for the trisubstituted triazines which promote nerve regeneration.

FIG. 2 is data from a high-content screen of neurite growth on Poly-L-lysine (PDL)/myelin. The data for 312 compounds are summarized for total neurite growth normalized to the PDL control. The first three bars show PDL (blue), PDL/myelin (red), and PDL/myelin/dbcAMP (green). Myelin strongly reduces growth, and this inhibition is overcome by cAMP. Straight lines drawn across data represent growth levels with dbcAMP, PDL, myelin, and 50% of the myelin level. The majority of compounds had little or no effect at the concentrations tested (5 μM is shown). Four compounds were found to increase growth substantially (more than two times growth on myelin). These four compounds are subsequently referred to as lead compounds.

FIG. 3 shows the results of quantitative analysis of neurite growth on CSPG substrates. Laminin (LN)-mediated growth was severely inhibited by the CSPG mixture. This inhibition on the LN/CSPG mixture (LC) was largely reversed by the conventional Protein Kinase C (cPKC) inhibitor Go6976 (LC/Go). Each of the four lead compounds was found to reverse CSPG-mediated inhibition as well as or better than the cPKC inhibitor. Asterisks identify compounds that were significantly different from LN/CSPG.

FIG. 4 shows that the lead compounds did not increase neurite growth on permissive substrates. CGNs were cultured on PDL or laminin (LN) substrates in the absence or presence of one of the four lead compounds, or on LN in the presence of Gö6976. Neurons grew neurites on PDL, and grew longer neurites on LN. None of the lead compounds increased neurite growth on either substrate, and the A05 compound (A5) slightly decreased growth. In contrast thereto, the cPKC inhibitor (Gö6976) significantly increased growth on LN, strongly suggesting that the lead compounds do not act by inhibiting cPKC. The failure of the compounds to increase neurite growth on permissive substrates strongly suggests that they act selectively to overcome inhibitory signals.

FIGS. 5A-D demonstrate that the lead compounds promoted growth of cortical neurons on CNS myelin. E15 mouse cortical neurons were cultured for three days on PDL (FIG. 5A), PDL/myelin (FIG. 5B), or PDL/myelin in the presence of lead compounds A05 (FIG. 5C) and C05 (FIG. 5D). The CNS myelin strongly inhibited cortical neurite growth, and this inhibition was overcome by the lead compounds. In FIG. 5C, the cultures were double-stained for neuronal β-tubulin (green) and Glial Fibrillary Acidic Protein (GFAP) (red). The cortical neurons were often seen to adhere to astrocytes (arrowheads), but neurite growth occurred on the myelin substrate in this assay.

FIGS. 6 A-E show that the lead compounds increased the growth of spinal neurons on CSPG substrates. E15 rat spinal neurons were cultured on LN, or on LN and CSPGs (LN/CSPG) for two days. They were stained for nuclei (blue) and neuronal beta-tubulin (green). Growth was strongly inhibited by the CSPG mixture (FIG. 6B). Growth was somewhat restored by the cPKC inhibitor Gö6976 (Go, FIG. 6C), and more so in the presence of the lead compounds (FIG. 6D). FIG. 6E shows quantitative data for all four lead compounds.

FIG. 7 shows that compound F05 increases growth of mature RGCs on CSPGs. P20 RGCs were cultured for 5 days on an inhibitory substrate (LN/CSPGs), in the absence (A) or presence (B) of compound F05 at 1 μM. F05 significantly improved axon growth on the inhibitory CSPG/LN substrate, as can be seen in the cumulative neurite length histogram (C). A second experiment gave similar results with F05, as well as the other 3 hits (A05, C05, H08; data not shown).

FIG. 8 shows that the lead compounds did not increase cAMP levels in CGNs. CGNs were cultured for two hours on PDL or on PDL/myelin in the absence or presence of the four lead compounds at 5 μM of forskolin (F) at the concentrations indicated in the chart. Forskolin increased neuronal cAMP levels by 100-250-fold, while the lead compounds produced no significant increases. Similar results were obtained with neurons cultured for two days (not shown). The graph shows the mean±range for two independent experiments.

FIG. 9 demonstrates that the lead compounds do not act by inhibiting cPKC. An in vitro PKC assay showed that Calphostin C inhibits cPKC, as expected, while concentrations of lead compounds that are optimally effective when added to cells have either little or no effect on cPKC when preincubated directly with the enzyme.

FIG. 10 shows that the lead compounds do not act by inhibiting Epidermal Growth Factor Receptor (EGFR) activity. An EGFR activity assay in A431 cells demonstrated that, as expected, PD168393 (PD) completely inhibited EGF-stimulated EGFR activation, but none of the lead compounds significantly affected this activation at concentrations from one to ten times those optimally effective in promoting neurite growth on inhibitory substrates.

FIGS. 11A-H show an experiment on regeneration of optic nerves in rats. One optic nerve was crushed in adult rats. Treatments included intraocular injection of BSA and application of DMSO (vehicle) to the crush site (11A, 11B), intraocular injection of a “survival cocktail” (growth factors and cAMP) with DMSO application to the crush site (11C,11D), intraocular injection of BSA with compound F05 applied to the crush site (11E, 11F), and intraocular injection of survival cocktail with compound F05 applied to the crush site (11G, 11H). Fluorescently labeled cholera toxin B subunit (CTB-Alexa488, green) was coinjected intraocularly to trace the paths of RGC axons. Two weeks later, the optic nerves were removed, sectioned, and stained for nuclei (blue). The arrowheads mark the approximate site of the crush lesion. While few axons in the first three conditions are present past the lesion site, a large number of axons can be seen at a substantial distance beyond the lesion in the F05/survival cocktail group (11G, 11H). Thus, F05 has been shown to promote regeneration of RGC axons, when they are allowed to survive.

FIG. 12 illustrates the chemical structure of four of the most promising compounds, A05, C05, F05 and H08.

FIGS. 13A-G illustrate the effects of F05 on spinal cord injury in mice. A and D show dorsal column axons in the spinal cord of a mouse expressing GFP in sensory neurons before injury. Black line in midline is a large artery. B,E. 6 hrs after a cut, axons have retracted from the lesion site (dotted lines). C,F. By 48 hrs, 1 axon has crossed the lesion site in the F05-treated animal (arrow, C), while axons remain retracted in the saline control (arrows, F). G. “Best axons” from all animals examined. For each lesion, the axon that either i) retracted the shortest distance behind the lesion, or ii) advanced the farthest distance past the lesion was analyzed, and these distances plotted as “crossing distance”. Negative numbers represent retraction from the lesion. While axons ended up retracted from the lesion in control animals at 48 hrs, F05-treated axons retracted less, and in 6 cases regenerated past the lesion (defined as more than 50 mm); p<0.02. Thus, a high percentage of lesions treated with F05 displayed axons that grew past the lesion midpoint, while this was true for only one control lesion.

FIG. 14 shows dose-response relationships for four lead compounds. CGNs were cultured on PDL (control) or on myelin in the presence of the 4 hit compounds at concentrations (in nM) shown. The Cellomics Kinetic Scan Reader (an automated microscope) was used to evaluate average neurite length per neuron in each condition. Growth on myelin in the absence of compounds was set at 100% (origin of y-axis). The black horizontal line represents growth on the control (PDL) substrate; thus growth at or above this line represents complete reversal of the myelin inhibition. Each compound completely reversed growth inhibition by the myelin. EC₅₀ concentrations were calculated using Igor Pro (Wavemetrics, Eugene, Oreg.) and were 15 nM (H08),

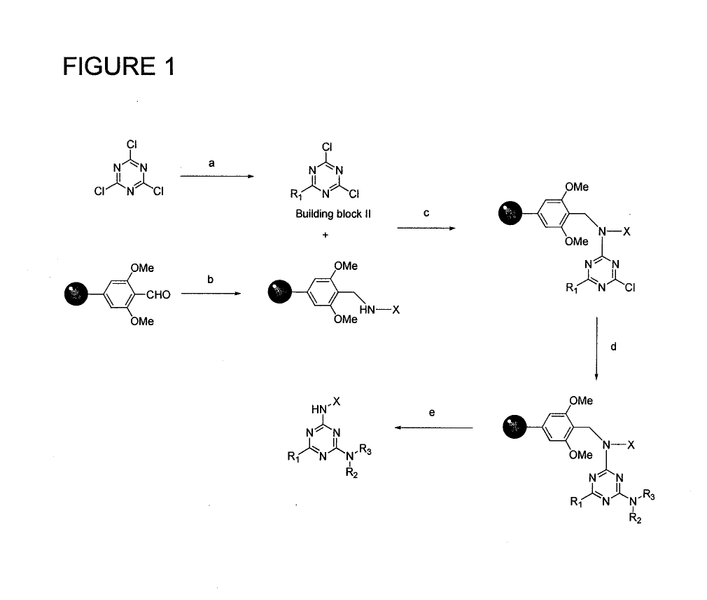

25 nM (A05), 9 nM (F05) and 14 nM (C05). N=2 experiments. Note log scale on x axis.

### DETAILED DESCRIPTION

Triazine is used as the linker library scaffold for the present compounds. Triazines are used because they are structurally similar to purine and pyrimidine, and they have demonstrated their biological activities in many applications. In particular, the triazine compounds used herein have many drug-like properties, including molecular weight of less than 500, cLogP of less than 5, etc. As the triazine scaffold has three-fold symmetry, it is readily possible to generate many diverse compounds. Furthermore, the starting material, triazine trichloride, and all of the required building blocks, which are amines, are relatively inexpensive. Because of its ease of manipulation and the low price of the starting material, triazine has elicited much interest as an ideal scaffold for a combinatorial library, resulting in several triazine libraries having been published in the literature. However, all of the reported library synthesis procedures, both for solid and solution phase chemistry, are based on sequential aminations using the reactivity differences of the three reaction sites. This library, by contrast, uses three different “building blocks” (see below).

The compounds described herein each contain a polyethylene glycol group as one of the substituents. This makes it possible to couple the compounds to a solid phase without further modification and potential loss of binding activity. The other substituent groups, R₁ and R₂, are substituted or unsubstituted alkyl or aryl/alkyl groups (R₁) or substituted or unsubstituted amines (R₂); R₃ and R₄ are each separately hydrogen, substituted or unsubstituted amine or substituted or unsubstituted C₁₋₁₀ alkyl groups, substituted or unsubstituted C₂₋₁₀ alkene groups, or C₂₋₁₀ alkynyl groups.

Some of the specific compounds identified in the screen contained phenyl- or chlorophenyl-based substituents at position R₁ (A, C or F) together with amine group 5 at position R₂ (A05, C05, F05) or group H at position R₁ and an amine group 8 at position R₂ (H08), with one of R₃ and R₄ being amine, and the other being hydrogen.

In the conventional method of triazine synthesis, the first substitution occurs at low temperatures while the second and third reactions require subsequently higher temperatures. This stepwise amination approach, however, is difficult to generalize for nucleophiles having differing reactivities. Thus, many byproducts may be generated together with the desired product.

The present reaction sequence solves the problem of byproducts using a straightforward synthetic pathway that can be used for the general preparation of a trisubstituted triazine library. The present process does not use selective amination, which requires careful monitoring of the reaction and purification steps. Instead, the present process uses three different kinds of building blocks to construct the library. The first amine (linker) is loaded onto an acid-labile aldehyde resin substrate, such as a mono- or di-methoxybenzaldehyde resin, by reductive amination. The second amine is added to cyanuric chloride to form a building block with the dichlorotriazine core structure. These two building blocks are then combined by amination of the first building block onto one of the chloride positions of the second building block. Any sequential over-amination on the other chloride position is efficiently suppressed by physical segregation from any other amine available on the solid support. The third building block, which can be a primary or secondary amine, then reacts with the last chloride position to produce the trisubstituted triazine. Since all reactions are orthogonal to each other, no further purification is required after cleavage of the final compound.

Generally, R₁ may be a C₁₋₂₀ alkyl group; unsubstituted phenyl or phenyl substituted with at least one of F, Cl, methoxy, ethoxy, trifluoromethyl, or C₁₋₆ alkyl; benzyl substituted with at least one of F, Cl, methoxy, ethoxy, trifluoromethyl, or C₁₋₆ alkyl; or a substituted or unsubstituted cycloaliphatic group.

R₂ may be a C₁₋₂₀ amino group, either straight chain, branched chain or heterocyclic, substituted with at least one of phenyl; phenyl substituted with at least one of F, Cl, methoxy, ethoxy, trifluoromethyl, or C₁₋₆ alkyl. R₃ and R₄ are individually hydrogen or substituted or unsubstituted C₁₋₁₀ alkyl, C₂₋₁₀ alkenyl, or C₂₋₁₀alkynyl.

As used herein, alkyl carbon chains, if not specified, contain from 1 to 20 carbon atoms, preferably from 1 to 14 carbon atoms, and are straight or branched.

As used herein an alkyl group substituent includes halo, haloalkyl, preferably halo lower alkyl, aryl, hydroxy, alkoxy, aryloxy, alkoxy, alkylthio, arylthio, aralkyloxy, aralkylthio, carboxy, alkoxycarbonyl, oxo, and cycloalkyl.

For the purposes of this description, “cyclic” refers to cyclic groups preferably containing from 3 to 19 carbon atoms, preferably 3 to 10 members, more preferably 5 to 7 members. Cyclic groups include hetero atoms, and may include bridged rings, fused rings, either heterocyclic, cyclic, or aryl rings.

The term “aryl” herein refers to aromatic cyclic compounds having up to 10 atoms, including carbon atoms, oxygen atoms, sulfur atoms, selenium atoms, etc. Aryl groups include, but are not limited to, groups such as phenyl, substituted phenyl, naphthyl, substituted naphthyl, in which the substituent is preferably lower alkyl or halogen. “Aryl” may also refer to fused rings systems having aromatic unsaturation. The fused ring systems can contain up to about 7 rings.

An “aryl group substituent” as used herein includes alkyl, cycloalkyl, cycloaryl, aryl, heteroaryl, optionally substituted with 1 or more, preferably 1 to 3, substituents selected from halo, haloalkyl, and alkyl, arylalkyl, heteroarylalkyl, alkenyl containing 1 to 2 double bonds, alkynyl containing 1 to 2 triple bonds, halo, hydroxy, polyhaloalkyl, preferably trifluoromethyl, formyl, alkylcarbonyl, arylcarbonyl, optionally substituted with 1 or more, preferably 1 to 3, substituents selected from halo, haloalkyl, alkyl, heteroarylcarbonyl, carboxyl, alkoxycarbonyl, aryloxycarbonyl, aminocarbonyl, alkylaminocarbonyl, dialkylaminocarbonyl, arylalkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, amido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfinyl, dialkylaminosulfonyl, and arylaminosulfonyl.

The term “arylalkyl” as used herein refers to an alkyl group which is substituted with one or more aryl groups. Examples of arylalkyl groups include benzyl, 9-fluorenylmethyl, naphthylmethyl, diphenylmethyl, and triphenylmethyl.

“Cycloalkyl” as used herein refers to a saturated mono- or multicyclic ring system, preferably of 3 to 10 carbon atoms, more preferably from 3 to 6 carbon atoms.

The term “heteroaryl” for purposes of the present application refers to a monocyclic or multicyclic ring system, preferably about 5 to about 15 members, in which at least one atom, preferably 1 to 3 atoms, is a heteroatom, that is, an element other than carbon, including nitrogen, oxygen, or sulfur atoms. The heteroaryl may be optionally substituted with one or more, preferably 1 to 3, aryl group substituents. Exemplary heteroaryl groups include, for example, furanyl, thienyl, pyridyl, pyrrolyl, N-methylpyrrolyl, quinolyinyl and isoquinolinyl.

The term “heterocyclic” refers to a monocyclic or multicyclic ring system, preferably of 3 to 10 members, more preferably 4 to 7 members, where one or more, preferably 1 to 3, of the atoms in the ring system is a heteroatom, i.e., an atom that is other than carbon, such as nitrogen, oxygen, or sulfur. The heterocycle may be optionally substituted with one or more, preferably 1 to 3, aryl group substituents. Preferred substituents of the heterocyclic group include hydroxy, alkoxy, halo lower alkyl. The term heterocyclic may include heteroaryl. Exemplary heterocyclics include, for example, pyrrolidinyl, piperidinyl, alkylpiperidinyl, morpholinyl, oxadiazolyl, or triazolyl.

The term “halogen” or “halide” includes F, Cl, Br, and I. This can include pseudohalides, which are anions that behave substantially similarly to halides. These compounds can be used in the same manner and treated in the same manner as halides. Pseudohalides include, but are not limited to, cyanide, cyanate, thiocyanate, selenocyanate, trifluoromethyl, and azide.

The term “haloalkyl” refers to a lower alkyl radical in which one or more of the hydrogen atoms are replaced by halogen, including but not limited to, chloromethyl, trifluoromethyl, 1-chloro-2-fluoroethyl, and the like. “Haloalkoxy” refers to RO— in which R is a haloalkyl group.

The term “sulfinyl” refers to —S(O)—. “Sulfonyl” refers to —S(O)₂—.

“Aminocarbonyl” refers to —C(O)NH₂.

The term “arylene” as used herein refers to a monocyclic or polycyclic bivalent aromatic group preferably having from 1 to 20 carbon atoms and at least one aromatic ring. The arylene group is optionally substituted with one or more alkyl group substituents. There may be optionally inserted around the arylene group one or more oxygen, sulfur, or substituted or unsubstituted nitrogen atoms, where the nitrogen substituent is alkyl.

“Heteroarylene” refers to a bivalent monocyclic or multicyclic ring system, preferably of about 5 to about 15 members, wherein one or more of the atoms in the ring system is a heteroatom. The heteroarylene may be optionally substituted with one or more aryl group substituents.

The term “library” refers to a collection of diverse compounds. In the present case, the library is based on a triazine scaffold.

### Experimental

Unless otherwise noted, materials and solvents were obtained from commercial suppliers and were used without further purification. Anhydrous tetrahydrofuran (THF) and 1-methyl-2-pyrrolidinone (NMP) from Acros were used as reaction solvents without any prior purification. PAL-aldehyde resin from Midwest Bio-Tech was used as the solid support. For the synthesis of building block q, general coupling reactions were performed through solution phase chemistry and were purified by flash column chromatography on Merck silica gel 60-PF₂₄₅. All products were identified by LCMS from Agilent Technology using a C18 column (20×4.0 mm), with a gradient of 5-95% CH₃CN (containing 1% acetic acid)-H₂O (containing 1% acetic acid) as eluant.

Thermal reactions were performed using a standard heat block from VWR Scientific Products using 4 ml vials. Resin filtration procedures were carried out using 70 microns PE frit cartridges from Applied Separations.

### Synthesis of the TG-Boc Linker (1)

Ten equivalents of 2,2′-(ethylenedioxy)bis(ethylamine) was dissolved in dichloromethane, and the solution was cooled down to −78° C. in a dry ice/acetone bath. One equivalent of di-tert-butyl dicarbonate was dissolved in dichloromethane and added to the solution of 2,2′-(ethylenedioxy)bis(ethylamine) dropwise over a period of three hours in a nitrogen gas atmosphere. The reaction mixture was allowed to stir for ten hours, followed by extraction with saturated NaCl solution. The organic layers were combined and dried over MgSO₄. The solvent was removed in vacuo.

### General Procedure for Preparing Building Block II Via Grignard Alkylation

A solution of 13.0 mmol of alkylmagnesiumhalide (Br/Cl) in 125 mL anhydrous THF was slowly added to a cooled (0° C.-5° C.) mechanically stirred THF solution of 2 grams, 10.93 mmol, of cyanuric chloride in 125 mL THF. The mixture was stirred at 0° C. for two hours. The reaction mixture was quenched with 50 mL of 1N HCl. The reaction mixture was then extracted with ethyl acetate and washed with water. The organic layers were combined and dried over MgSO₄. The solvent was removed in vacuo.

Loading of Amine onto PAL Resin Via Reductive Amination

To a suspension of 1.0 g, 1.1 mmol, 4-formyl-3,5-dimethoxyphenoxymethyl-functionalized polystyrene resin (PAL) in 40 mL THF was added 5.5 mmol of a primary amine, followed by the addition of 0.9 mL of acetic acid. After shaking the mixture at room temperature for one hour, 1.63 g, 7.7 mmol of NaBH(OAc)₃ was added, and the reaction continued with shaking at room temperature for eight hours. Using a PE frit cartridge, the solvents and excess reagents were filtered out and washed with DMF, MC and MeOH (20 mL×3), ending with a final washing with MC and dried under nitrogen gas.

### Resin Capture of Triazine Scaffold Via Amine Substitution

To a suspension of 125 mg, 0.132 mmol of the PAL-resin-bound amine in 2.5 mL THF was added 125 mg of one of 4,6-dichloro-[1,3,5]-triazine-2-yl-4-methoxy-benzyl-amine; 2-benzylsulfinyl-4,6-dichloro-[1,3,5]-triazine; or 2,4-dichloro-alkyl/aryl-[1,3,5]-triazine, followed by addition of 0.15 mL of diispropylethylamine. The reaction was placed into a heating block set tat 60° C. for 2.5 hours. The solvents and excess reagents were filtered through a PE frit cartridge and washed with DMF, DCM, MeOH (3 mL×3), consecutively, ending with a final washing with 3 mL DCM, and dried under nitrogen gas.

### Final Amination and Product Cleavage Reaction

To a suspension of 10 mg, 11 μmol, of the resin in 0.25 NMP was added 0.2 mmol of an amine, followed by the addition of 0.25 mL n-butanol and 30 microliters, 0.22 mmol diisopropylamine. The reaction was placed into a heating block set at 120° C. for three hours. The excess reagents were filtered through a PE frit cartridge and washed with DMF, DCM, MeOH (1 mL×3), consecutively, ending with a final washing with 1 mL DCM. The resin was dried in vacuum. The product cleavage reaction was performed using 10% trifluoroacetic acid (TFA) on 1 mL dichloromethane for one hour at room temperature and washed with 0.5 mL dichloromethane.

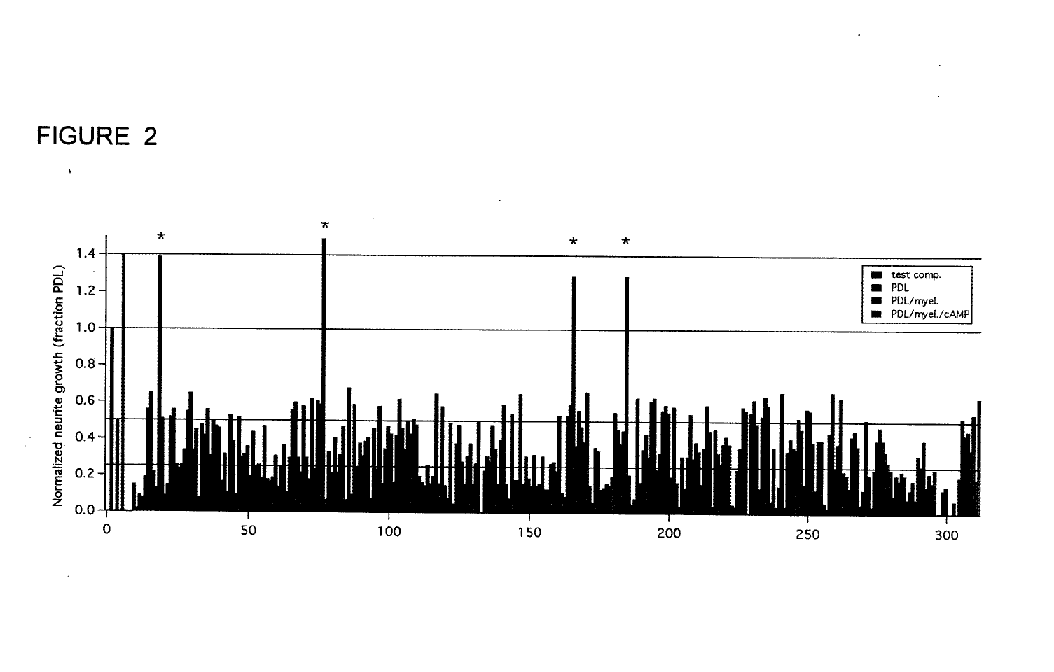

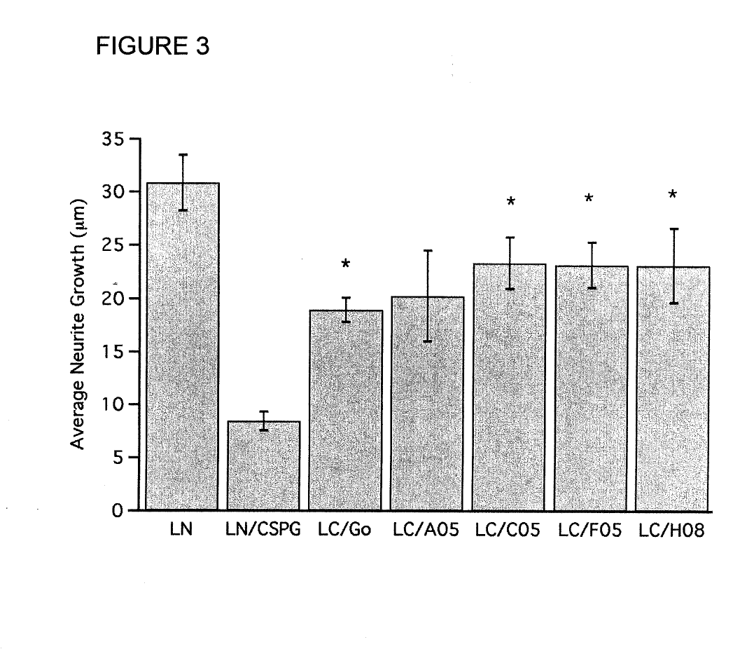

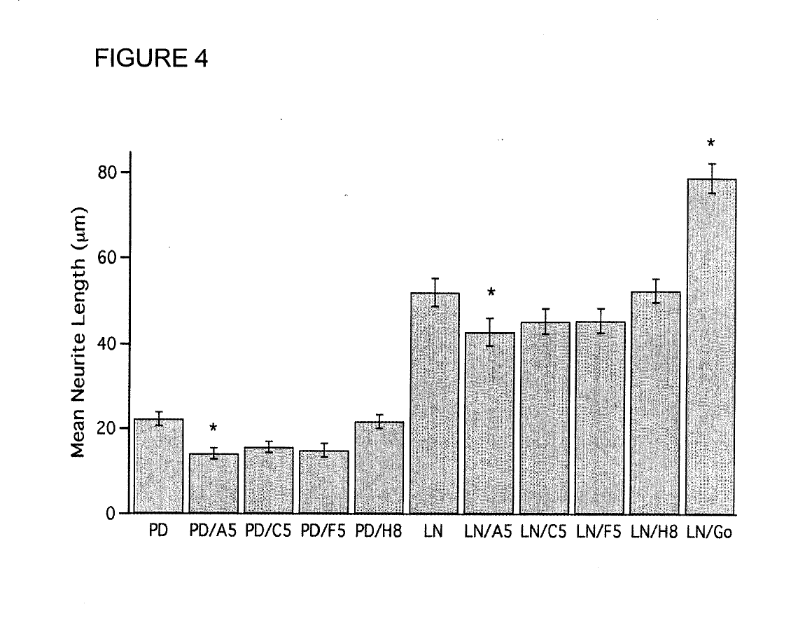

The triazine compounds were added to cultures of primary cerebellar granule neurons (CGNs) to test their ability to promote neurite growth on an inhibitory substrate (PDL/central nervous system myelin). Of the more than 400 compounds tested, four, all from plate AA4 (A05, C05, F05 and H08), were able to promote substantial neurite growth in this condition (see asterisks in “Neuron data”, FIG. 2). The compounds can promote growth on other inhibitory substrates as well. When a mixture of inhibitory chondroitin sulfate proteoglycans (CSPGs) on PDL was used as a substrate, neurite growth of CGNs was increased by each compound, as shown in FIG. 3. The compounds evidently act by overcoming inhibitory signals. Though they increase growth on inhibitory substrates, they do not increase growth on normally permissive substrates like laminin or poly-D-lysine (PDL, FIG. 4).

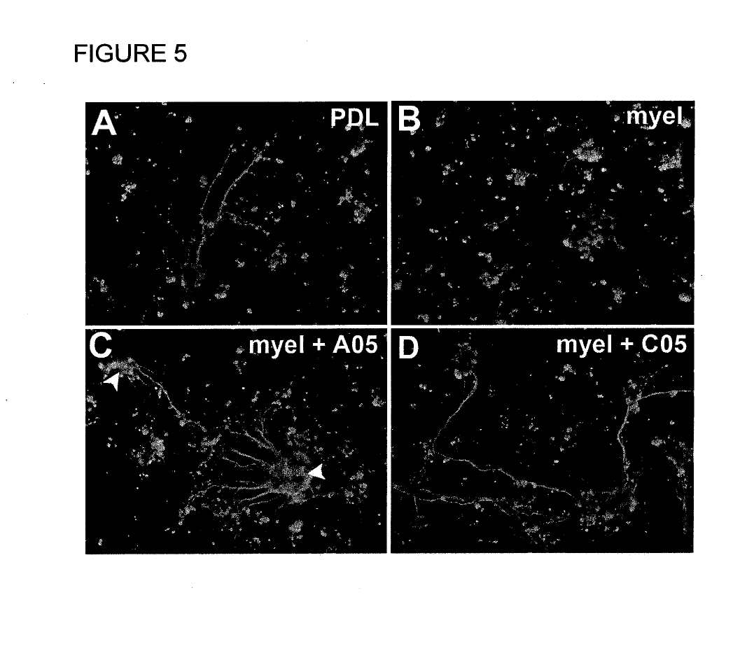

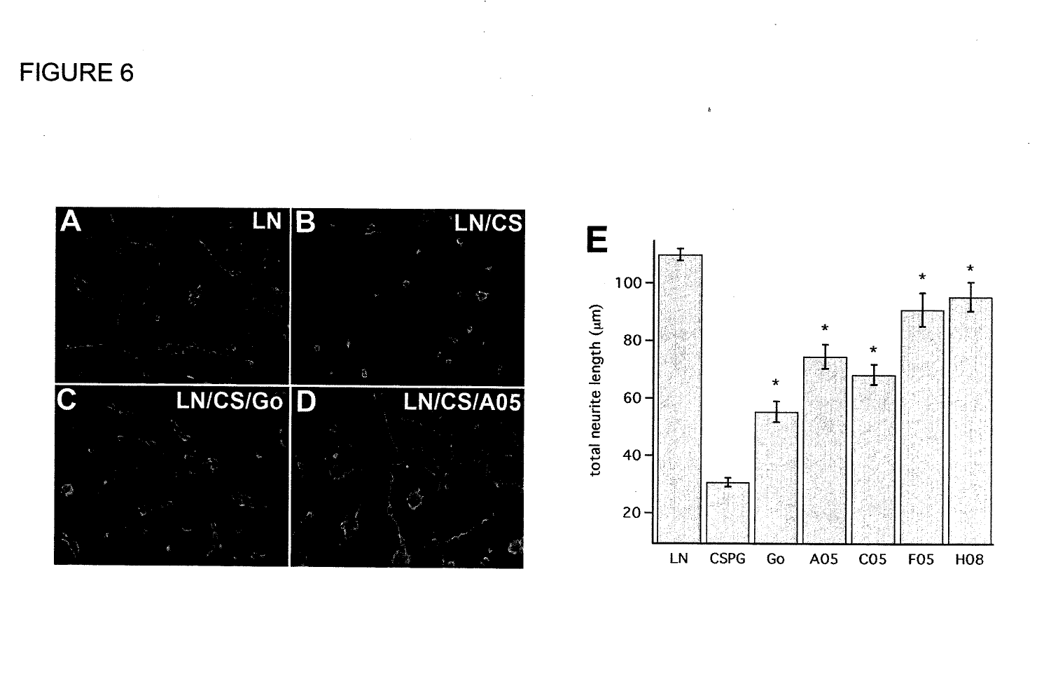

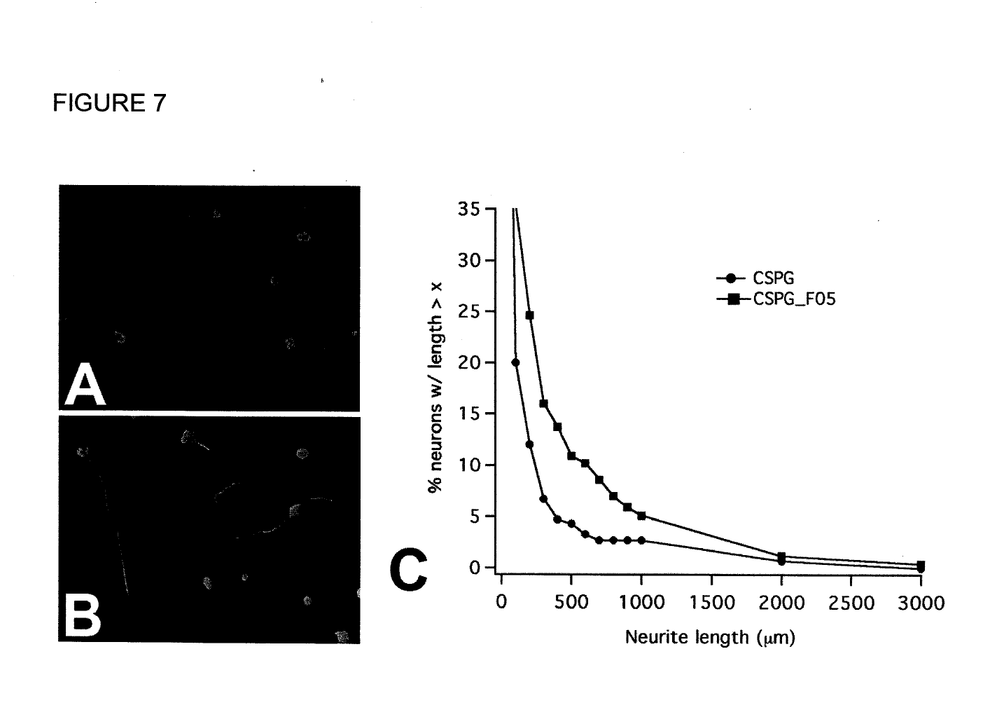

It was also found that the compounds are active on a variety of neuronal types from the central nervous system, including cortical neurons (FIG. 5), spinal neurons (FIG. 6) and mature retinal ganglion neurons (FIG. 7).

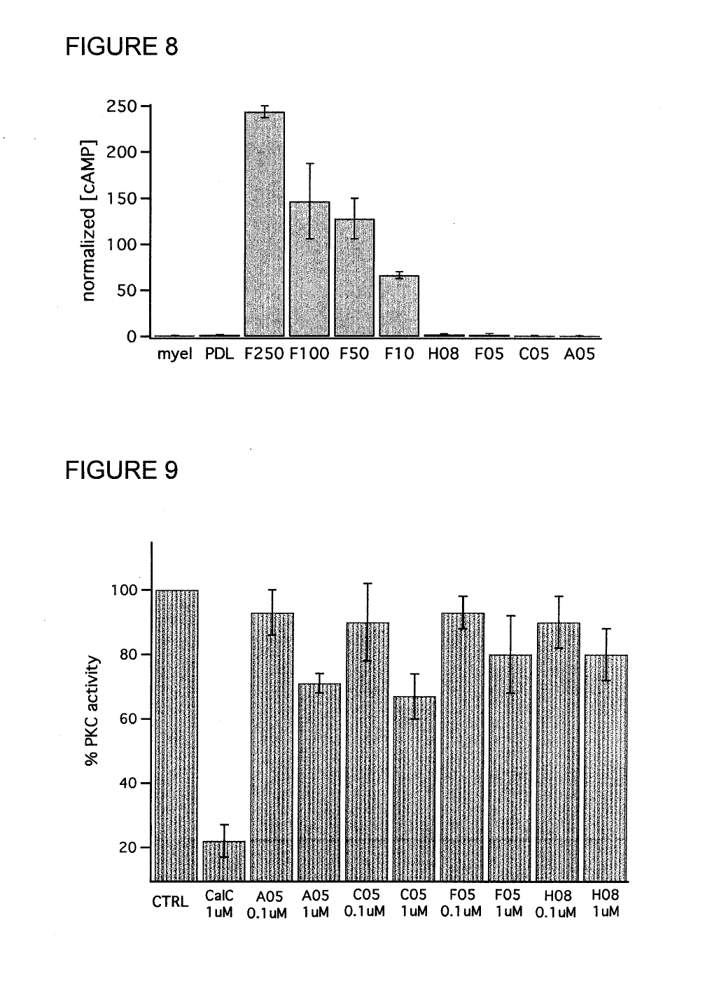

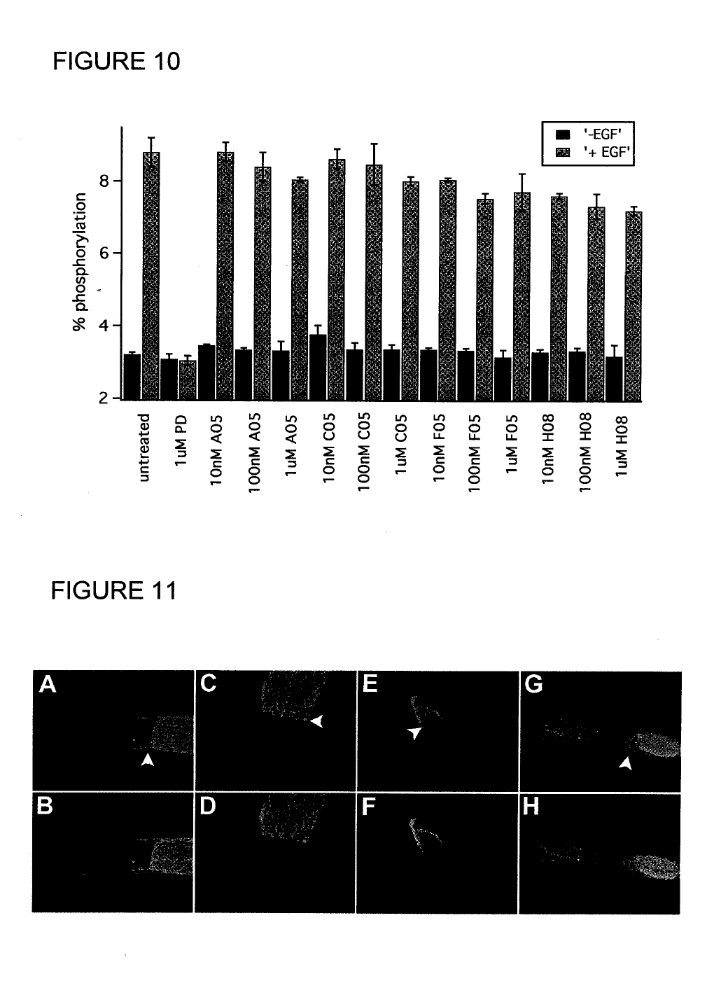

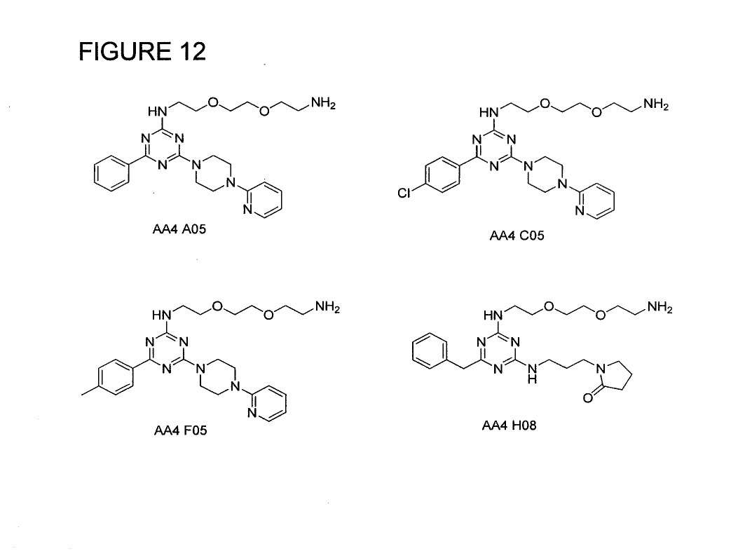

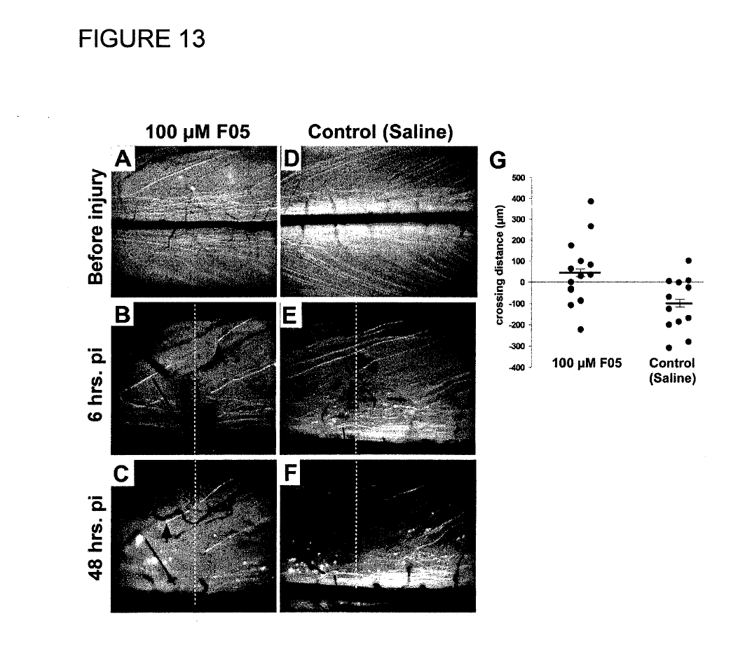

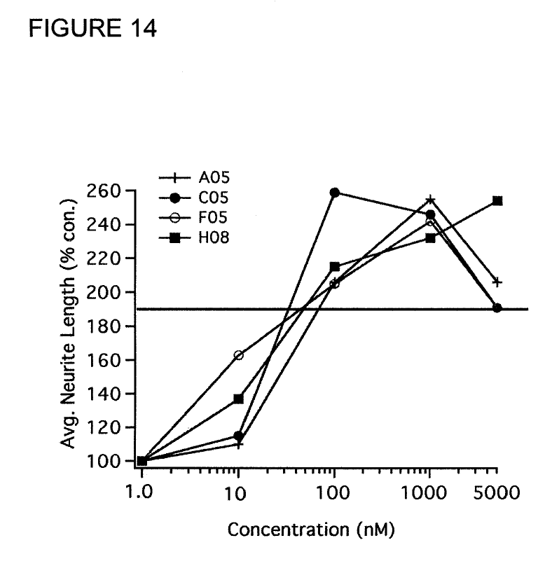

Currently, the molecular targets of the compounds are not known, although the data suggest that the compounds do not promote growth by increasing cAMP (FIG. 8), inhibiting protein kinase C (FIG. 4 and data not shown), or inhibiting the EGF receptor (FIG. 10). Each of these mechanisms is thought to be involved in overcoming regeneration inhibition by CSPGs and/or myelin. Preliminary data using direct application of compound F05 to the axons of crushed rat optic nerve suggest that F05 can promote regeneration of adult central nervous system axons in vivo (FIG. 11).

Examples of compounds included in the present library are the following:

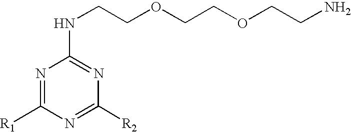

wherein R₁ and R₂ are as shown in the following tables.

TABLE 1

| R1 | Aryl/alkyl |
|---|---|
| A | 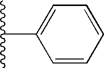 |
| B | 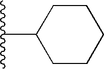 |
| C | 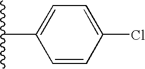 |
| D | 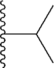 |
| E | 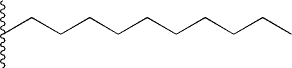 |
| F | 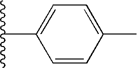 |
| G | 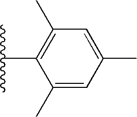 |
| H | 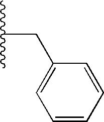 |

TABLE 2

| R2 | Amine |
|---|---|
| 1 | 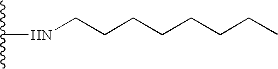 |
| 2 | 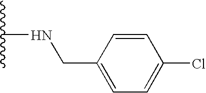 |
| 3 | 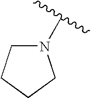 |
| 4 | 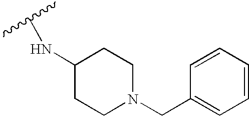 |
| 5 | 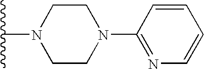 |
| 6 | 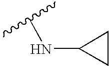 |
| 7 | 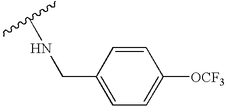 |
| 8 | 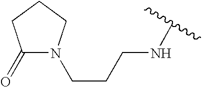 |
| 9 | 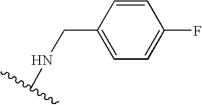 |
| 10 | 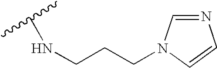 |

Scheme 1. Synthetic scheme for the AA4 library. General scheme for orthogonal synthesis reagents and conditions: (a) R1MgX (1.2 eq), THF, 0 oC, 2 h (1/1) (b) TG-Boc linker (1; 5 eq), 2% acetic acid in THF, rt., 1 h, followed by NaB(OAc)₃H (7 eq), rt., 12 h. (c) Building block II (4 eq) in THF 60° C., 1 h, DIEA. (d) R₂R₃′NH, DIEA, NMP:n-BuOH=1:1, 120° C., 3 h. (e) 10% TFA in dichloromethane, rt., 1 h.

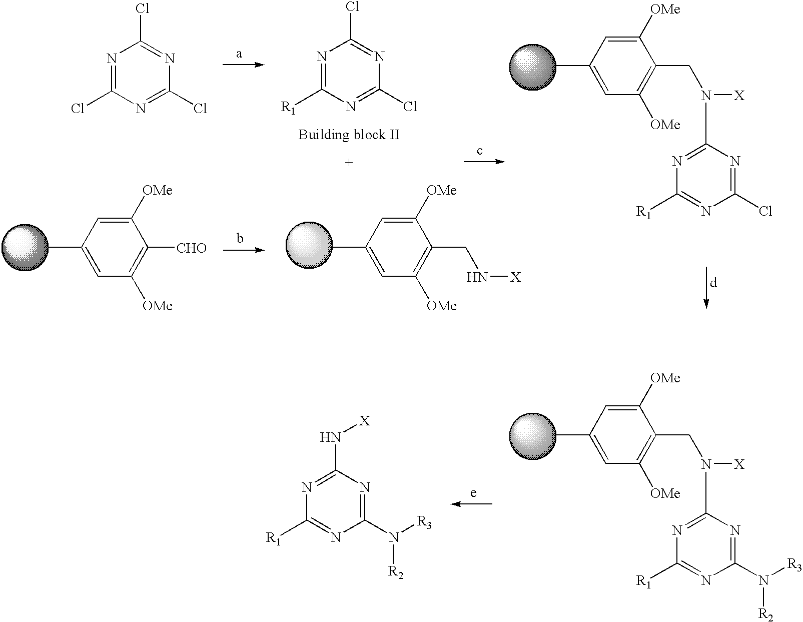

Effective compounds identified from the library can be used in pharmaceutical compositions, for example, for the treatment of nerve injury, e.g. traumatic brain injury, stroke spinal cord injury, multiple sclerosis, or diseases that affect the central nervous system or optic nerve.

Pharmaceutical compositions as described herein can be administered by any convenient route, including parenteral or intravenous. Delivery is generally directly to the site of injury. The dosage administered depends upon the age, health, and weight of the recipient, nature of concurrent treatment, if any, and the nature of the effect desired.

Compositions within the scope of this application include all compositions wherein the active ingredient is contained in an amount effective to achieve its intended purpose. While individual needs vary, determination of optimal ranges of effective amounts of each compound is within the skill of the art. Typical dosages comprise 0.01 to 100 mg/kg body weight. The preferred dosages comprising 0.1 to 100 mg/kg body weight. The most preferred dosages comprise 1 to 50 mg/kg body weight.

Pharmaceutical compositions for administering the active ingredients preferably contain, in addition to the pharmacologically active compound, suitable pharmaceutically acceptable carriers comprising excipients and auxiliaries which facilitate processing of the active compounds into preparations which can be used pharmaceutically. Preferably, the preparations contain from about 0.01 to about 99 percent by weight, preferably from about 20 to 75 percent by weight, active compound(s), together with the excipient. For purposes of the present discussion, all percentages are by weight unless otherwise indicated. In addition to the following described pharmaceutical composition, the compounds described herein can be formulated as inclusion complexes, such as cyclodextrin inclusion complexes.

The pharmaceutically acceptable carriers include vehicles, adjuvants, excipients, or diluents that are well known to those skilled in the art and which are readily available. It is preferred that the pharmaceutically acceptable carrier be one which is chemically inert to the active compounds and which has no detrimental side effects or toxicity under the conditions of use.

The choice of carrier is determined partly by the particular active ingredient, as well as by the particular method used to administer the composition. Accordingly, there is a wide variety of suitable formulations of the trisubstituted triazines described herein. Formulations can be prepared for parenteral, subcutaneous and intravenous administration

Suitable formulations for parenteral administration include aqueous solutions of the active compounds in water-soluble form, such as water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension, including, for example, sodium carboxymethyl cellulose, sorbitol, and/or dextran. Optionally, the suspension may also contain stabilizers.

Other pharmaceutically acceptable carriers for the active ingredients are liposomes, pharmaceutical compositions in which the active ingredient is contained either dispersed or variously present in corpuscles consisting of aqueous concentric layers adherent to lipid layers. The active ingredient may be present both in the aqueous layer and in the lipid layer, inside or outside, or, in any event, in the nonhomogeneous system generally known as a liposomic suspension.

The hydrophobic layer, or lipid layer, generally, but not exclusively, comprises phospholipids such as lecithin and sphingomyelin, steroids such as cholesterol, more or less ionic surface active substances such as dicetyl phosphate, stearylamine, or phosphatidic acid, and/or other materials of a hydrophobic nature.

The compounds may also be formulated for transdermal administration, for example in the form of transdermal patches so as to achieve systemic administration.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compounds can be administered in a physiologically acceptable diluent in pharmaceutical carriers, such as a sterile liquid or mixture of liquids, including water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,3-dioxolane-4-methanol, ethers such as poly(ethylene glycol) 400, oils, fatty acids, fatty acid esters or glycerides, or acetylated fatty acid glycerides, without the addition of a pharmaceutically acceptable surfactants, such as soap or a detergent, suspending agent, such as carbomers, methylcellulose, hydroxypropylmethylcellulose, or carboxymethylcellulose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Fatty acids can be used in parenteral formulations, including oleic acid, stearic acid, and isostearic acid. Ethyl oleate and isopropyl myristate are examples of suitable fatty acid esters. Suitable salts for use in parenteral formulations include fatty alkali metal, ammonium, and triethanolamine salts, and suitable detergents include cationic detergents such as dimethyl dialkyl ammonium halides, and alkyl pyridimium halides; anionic detergents such as dimethyl olefin sulfonates, alkyl, olefin, ether, and monoglyceride sulfates and sulfosuccinates; polyoxyethylenepolypropylene copolymers; amphoteric detergents such as alkyl-beta-aminopropionates and 2-alkyl-imidazoline quaternarry ammonium salts; and mixtures thereof.

Parenteral formulations typically contain from about 0.5 to 25% by weight of the active ingredient in solution. Suitable preservatives and buffers can be used in these formulations. In order to minimize or eliminate irritation at the site of injection, these compositions may contain one or more nonionic surfactants having a hydrophilic-lipophilic balance (HLB) in a range from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be present in unit dose or multiple dose sealed containers, such as ampules and vials, and can be stored in a freeze-dried (lyophilized) condition requiring only the addition of the sterile liquid carrier, e.g., water, for injections immediately prior to use. Extemporaneous injection solutions and suspensions can be prepared from sterile powders, granules, and tablets of the kind previously described.

The active ingredients can be used as functionalized congeners for coupling to other molecules, such as amines and peptides. The use of such congeners provides for increased potency, prolonged duration of action, and prodrugs. Water solubility is also enhanced, which allows for reduction, if not complete elimination, of undesirable binding to plasma proteins and partition in to lipids. Accordingly, improved pharmacokinetics can be realized.

Any number of assays well known in the art may be used to test whether a particular compound that is suspected of promoting nerve regeneration is actually capable of promoting nerve regeneration. The assays described herein can be used to determine the nerve regenerating activity of a compound without undue experimentation.

In determining the dosages of the compound to be administered, the dosage and frequency of administration is selected in relation to the pharmacological properties of the specific active ingredients. Normally, at least three dosage levels should be used. In toxicity studies in general, the highest dose should reach a toxic level but be sublethal for most animals in the group. If possible, the lowest dose should induce a biologically demonstrable effect. These studies should be performed in parallel for each compound selected.

Additionally, the ID₅₀ level of the active ingredient in question can be one of the dosage levels selected, and the other two selected to reach a toxic level. The lowest dose used should be one that does not exhibit a biologically demonstrable effect. The toxicology tests should be repeated using appropriate new doses calculated on the basis of the results obtained. Young, healthy mice or rats belonging to a well-defined strain are the first choice of species, and the first studies generally use the preferred route of administration. Control groups given a placebo or that are untreated are included in the tests. Tests for general toxicity, as outlined above, should normally be repeated in another non-rodent species, e.g., a rabbit or dog. Studies may also be repeated using alternate routes of administration.

Single dose toxicity tests should be conducted in such a way that signs of acute toxicity are revealed and the mode of death determined. The dosage to be administered is calculated on the basis of the results obtained in the above-mentioned toxicity tests. It may be desired not to continue studying all of the initially selected compounds. Data on single dose toxicity, e.g., ID₅₀, the dosage at which half of the experimental animals die, is to be expressed in units of weight or volume per kg of body weight and should generally be furnished for at least two species with different modes of administration. In addition to the ID₅₀ value in rodents, it is desirable to determine the highest tolerated dose and/or lowest lethal dose for other species, i.e., dog and rabbit.

When a suitable and presumably safe dosage level has been established as outlined above, studies on the drug's chronic toxicity, its effect on reproduction, and potential mutagenicity may also be required in order to ensure that the calculated appropriate dosage range will be safe, also with regard to these hazards.

Pharmacological animal studies on pharmacokinetics revealing, e.g., absorption, distribution, biotransformation, and excretion of the active ingredient and metabolites are then performed. Using the results obtained, studies on human pharmacology are then designed. Studies of the pharmacodynamics and pharmacokinetics of the compounds in humans should normally be performed in healthy subjects using the routes of administration intended for clinical use, and can be repeated in patients. The dose-response relationship when different doses are given, or when several types of conjugates or combinations of conjugates and free compounds are given, should be studied in order to elucidate the dose-response relationship (dose vs. plasma concentration vs. effect), the therapeutic range, and the optimum dose interval. Also, studies on time-effect relationship, e.g., studies into the time-course of the effect and studies on different organs in order to elucidate the desired and undesired pharmacological effects of the drug, in particular on other vital organ systems, should be performed.

The presently described substituted triazines are then ready for clinical trials to compare the efficacy of the compounds to existing therapy. A dose-response relationship to therapeutic effect and for side effects can be more finely established at this point.

The amount of the compounds to be administered to any given patient must be determined empirically, and will differ depending upon the condition of the patients. Relatively small amounts of the active ingredient can be administered at first, with steadily increasing dosages if no adverse effects are noted. Of course, the maximum safe dosage as determined by routine animal toxicity tests should never be exceeded.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should be and are intended to be comprehended within the meaning and range of equivalents of the disclosed embodiments.

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and “means for . . . ”, or any method step language, as may be found in the specification above and/or in the claims below, followed by a functional statement, are intended to define and cover whatever structural, physical, chemical, or electrical element or structure, or whatever method step, which may now or in the future exist which carries out the recited functions, whether or not precisely equivalent to the embodiment or embodiments disclosed in the specification above, i.e., other means or steps for carrying out the same function can be used; and it is intended that such expressions be given their broadest interpretation.

1. A substituted triazine compound of the formula 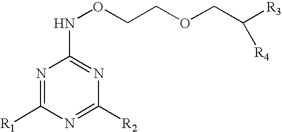
wherein R₁ is substituted or unsubstituted C₁₋₁₀ alkyl, C₂₋₁₀ alkenyl, C₂₋₂₀ alkynyl, alkenyl or aryl/alkyl; R₂ is substituted or unsubstituted amine; R₃ and R₄ are individually hydrogen, substituted or unsubstituted amine, substituted or unsubstituted C₁₋₁₀ alkyl, C₂₋₁₀ alkene, and C₂₋₂₀ alkynyl.

2. The compound according to claim 1, wherein R₁ is selected from the group consisting of: 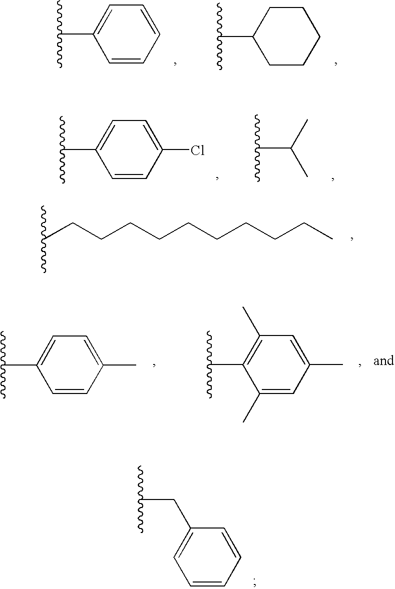
and R₂ is selected from the group consisting of:

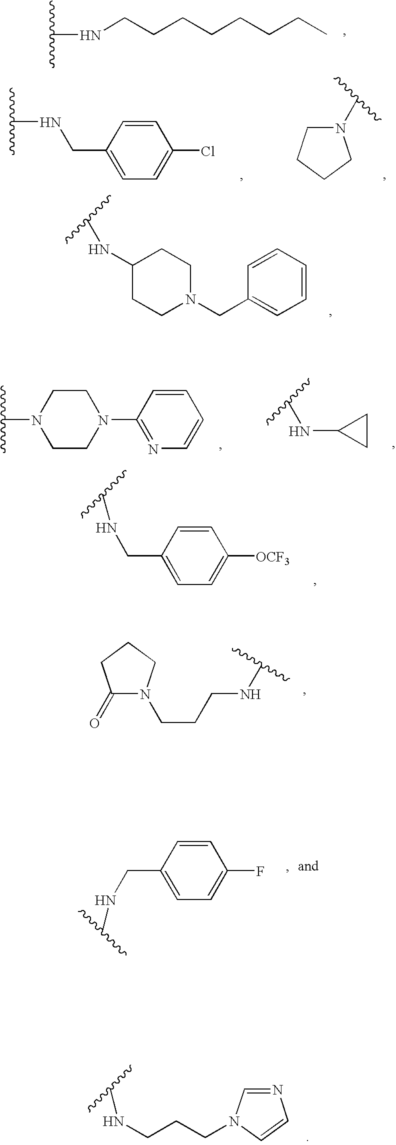

3. The compound according to claim 1 wherein R₁ is

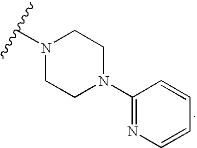

4. The compound according to claim 1 wherein R₁ is benzyl or phenyl or phenyl substituted with at least one of Cl, Br, C₁₋₄ alkyl, C₂₋₄ alkenyl or C₂₋₄ alkynyl.

5. The compound according to claim 1 of the formula

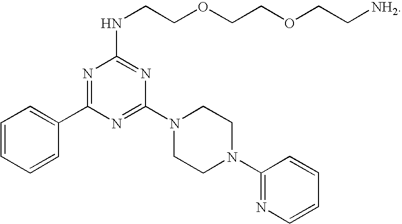

6. The compound according to claim 4 wherein R₂ is

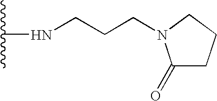

7. A pharmaceutical composition comprising a compound of claim 1, or a pharmaceutically acceptable salt thereof.

8. The pharmaceutical composition of claim 7 of formula 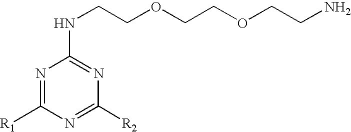
wherein R₁ is a substituted or unsubstituted phenyl group, and R₂ is a substituted or unsubstituted amine.

9. The pharmaceutical composition of claim 8 wherein R2 comprises at least one heterocyclic nitrogen-containing ring.

10. The pharmaceutical composition of claim 9 comprising a compound selected from the group consisting of 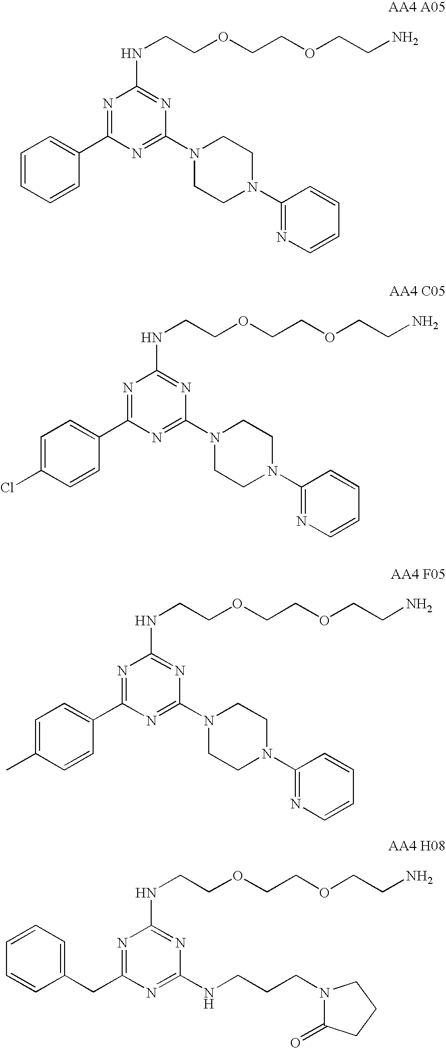
or a pharmaceutically acceptable salt thereof.

11. A compound library comprising two or more compounds of claim 1.

12. A compound library comprising at least 100 compounds of claim 1.

13. A method for increasing the growth of axons, dendrites, sprouts, branches, and combinations thereof, comprising administering to a patient in need thereof an effective amount of a compound or pharmaceutical composition according to claim 1 to increase the growth of neurites and axons.

14. The method according to claim 13, wherein the patient is suffering from traumatic brain injury, stroke, spinal cord injury, multiple sclerosis, or a disease that affects the central nervous system or optic nerve.

15. The method according to claim 14, wherein the compound is selected from the group consisting of:

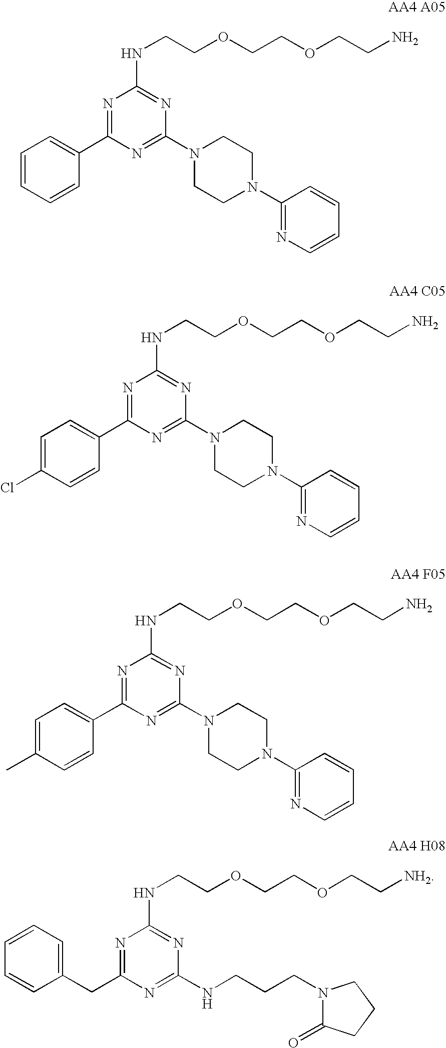

16. A method of identifying compounds effective for increasing the growth of axons, dendrites, sprouts, branches, and combinations thereof, the method comprising screening compounds of the compound library of claim 11 for stimulation of neural growth.

17. The method of claim 16 wherein the screening is carried out in vitro.

18. The method of claim 16 wherein the screening is carried out in vivo.

19. (canceled)

20. (canceled)

21. (canceled)
